(19) United States
(12) Patent Application Publication
(10) Pub. No.: US 2003/0185784 A1
(43) Pub. Date: Oct. 2, 2003

(54) Novel active agent, composition containing it and use in the cosmetic, dermocosmetic, dermopharmaceutical or pharmaceutical field or on woven nonwoven supports

(75) Inventors: Corrine Stoltz, Thiais (FR); Herve Geoffroy, Lesigny (FR)

Correspondence Address: YOUNG & THOMPSON, 745 SOUTH 23RD STREET 2ND FLOOR, ARLINGTON, VA, 22202

(73) Assignee: SOCIETE D'EXPLOITATION DE PRODUITS POUR LES INDUSTRIES CHIMIQUES SEPPIC, PARIS CEDEX (FR)

(21) Appl. No.: 10/394,246

(22) Filed: Mar. 24, 2003

Related U.S. Application Data
(62) Division of application No. 09/826,963, filed on Apr. 6, 2001.

(30) Foreign Application Priority Data
Apr. 6, 2000 (FR) 0004412

(51) Int. Cl. A61K 7/06; A61K 7/11; A61K 7/075; A61K 7/08

(52) U.S. Cl. 424/70.22; 424/70.12

(57) ABSTRACT

Composition, characterized in that it comprises, as active ingredient, at least one compound of formula (I):

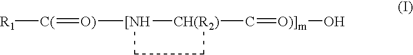

or its salts, in which R₁ represents the characterizing chain of a fatty acid comprising from 8 to 30 carbon atoms, R₂ represents the characterizing chain of an amino acid and m is between 1 and 5, and at least one compound of formula (II):

ZO—(CH₂)₃—NH—C(═O)—CH(OH)—C(CH₃)₂—CH₂—OH (II)

in which Z represents a hydrogen atom, or a radical derived from the phosphorus of formula:

P(R₃O)(OM)(═O)—

in which OM represents a free or salified OH radical, and R₃ is a radical derived from alkoxylated polysiloxanes. Use of the said composition for the preparation of cosmetic, dermocosmetic, dermopharmaceutical or pharmaceutical compositions, or on woven or nonwoven supports. Compositions thus prepared.

The subject of the present invention is a novel cosmetic composition of compounds having a lipoamino acid structure, with soothing activity.

In modern life, the hair is attacked by various factors external to the human body, such as atmospheric pollution, ultraviolet radiation, whether natural or artificial, or other stresses, whether mechanical or chemical.

Because these problems are increasingly taken into account, in particular in highly urbanized areas, the hair protection aspect has become preponderant in the search for novel cosmetic products.

The applicant has developed the novel concept of multiprotective and thermoactive hair active agent, for stressed hair, in response to the attacks or to the sensations of attack felt by the subject and, more particularly, in response to damage of free radical origin. It has discovered that the stress of the hair system induces the formation of free radicals and of lipid peroxides. At the end of a cascade of free radical reactions (Fenton reaction) which adversely affects protein metabolism, cell division is slowed down and the hair fibre is damaged at the level of the keratin of the hair and of its root. The hair then becomes brittle, flaky and dull. For effective protection of the entire hair, it is therefore essential, for a hair active agent, to be able to reduce the amount of lipid peroxides formed at the surface of the hair.

Because of their amphiphilic structure, compounds having a lipoamino acid structure, such as those described in international patent applications published under the numbers WO 92/20647, WO 92/21318, WO 94/26694 and WO 94/27561, are particularly useful biological vectors because they regulate skin physiology. They are therefore found to be appropriate for numerous applications, in particular in cosmetics.

Panthenol or 2,4-dihydroxy-N-(3-hydroxypropyl)-3,3-dimethylbutanamide of formula:

HO—(CH₂)₃—NH—C(═O)—CH(OH)—C(CH₃)₂—CH₂—OH

is a compound which is commonly used in the treatment of the hair and the scalp.

However, the applicant has found that compositions comprising, as active ingredients, the combination of lipoamino acids with panthenol or its derivatives, possess at the same time an anti-free-radical activity, a cell-division-stimulating activity, exacerbated by heat, and a keratin-protecting activity preventing the formation of flakes. The applicant has also found that this activity is the result of synergy derived from the combination of these two families of active ingredients.

The subject of the invention is a composition, characterized in that it comprises, as active ingredient, at least one compound of formula (I):

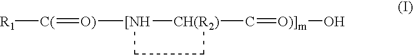

or its salts, in which R₁ represents the characterizing chain of a saturated or unsaturated, linear or branched fatty acid comprising from 8 to 30 carbon atoms, R₂ represents the characterizing chain of an amino acid and m is between 1 and 5, and at least one compound of formula (II):

ZO—(CH₂)₃—NH—C(═O)—CH(OH)—C(CH₃)₂—CH₂—OH (II)

in which Z represents a hydrogen, atom, or a radical derived from phosphorus

P(═O)(R₃O)(OM)—

in which OM represents a free or salified OH radical, either in the form of an alkali metal salt such as the -sodium salt or the potassium salt, or in the form of an ammonium salt or in the form of a salt of an amino alcohol such as a (2-hydroxyethyl)ammonium salt, and R₃ represents a radical derived from alkoxylated polysiloxanes.

The compound of formula (I) present in the composition which is the subject of the present invention may be in a free acid form or in a partially or completely salified form. When the compound of formula (I) is in salified form, this includes in particular alkali metal salts such as the sodium, potassium or lithium salts, alkaline-earth metal salts such as the calcium, magnesium or strontium salts; an ammonium salt or a salt of an amino alcohol such as the (2-hydroxyethyl)ammonium salt. This may also include metal salts such as the divalent zinc or manganese salts, the trivalent iron, lanthanum, cerium or aluminium salts.

The expression “characterizing chain” used to define the radicals R₁ and R₂ denotes the nonfunctional principal chain of the fatty acid or of the amino acid considered.

Thus, for a fatty acid corresponding to the general formula R₁—C(═O)—OH, the characterizing chain will be the chain represented by R₁. The radical R₁ represents in particular a radical comprising from 8 to 22 carbon atoms which is chosen from the octyl, nonyl, decyl, undecyl, dodecyl, tridecyl, tetradecyl, pentadecyl, hexadecyl, heptadecyl, octadecyl, nonadecyl, eicosyl, uneicosyl, docosyl, heptadecenyl, eicosenyl, uneicosenyl, docosenyl or heptadecadienyl or decenyl radicals.

According to a first particular aspect, the subject of the invention is a composition as described above for which, in formula (I), the group R₁—C(═O)— comprises from 8 to 22 carbon atoms and represents in particular one of the radicals octanoyl (caprylyl), decanoyl, undecylenoyl, dodecanoyl (lauroyl), tetradecanoyl (myristyl), hexadecanoyl (palmitoyl), octadecanoyl (stearyl), eicosanoyl (arachidoyl), docosanoyl (behenoyl), 8-octadecenoyl (oleyl), eicosenoyl (gadoloyl), 13-docosenoyl (erucyl), 9,12-octadecadienoyl (linoleoyl) or 9,12,15-octadecatrienoyl (linolenoyl).

According to a first preferred variant of the present invention, in formula (I), the fragment R₁—C(═O) comprises from 12 to 18 carbon atoms.

For an amino acid represented by the general formula (IIIa):

H₂N—CH(R₂)—C(═O)—OH (IIIa)

as for a cyclic amino acid represented by the formula (IIIb):

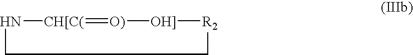

the characterizing chain will be the chain represented by R₂.

R₂ represents in particular the characterizing chain of an amino acid chosen from glycine, alanine, serine, aspartic acid, glutamic acid, valine, threonine, arginine, lysine, proline, leucine, phenylalanine, isoleucine, histidine, tyrosine, tryptophan, asparagine, cysteine, cystine, methionine, hydroxyproline, hydroxylysine or ornithine.

According to a second particular aspect, the subject of the invention is a composition as defined above, comprising at least one compound of formula (I) chosen from the N-acylated derivatives of glutamic acid, aspartic acid, alanine or glycine.

In formula (I) as defined above, m is in particular a decimal number less than or equal to 2, more particularly less than or equal to 1.4; that is equal to 1.

According to a third particular aspect, the subject of the invention is a composition as described above, comprising a single compound of formula (I).

According to a fourth particular aspect, the subject of the invention is a composition as described above, comprising a mixture of compounds of formula (I), and in particular, either a mixture of compounds of formulae (I) all comprising the same fragment R₁—C(═O) or else a mixture of compounds of formulae (I) in which m is equal to 1 and all comprising the same fragment

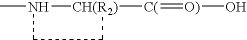

The compounds of formulae (I) are generally obtained by N-acylation of compounds of formulae (IIIa) or (IIIb), as defined above, or of their salts. When this includes a mixture of compounds of formulae (I), it is for example obtained by N-acylation of the mixture of amino acids resulting from the total or partial hydrolysis of proteins of any origin. These proteins may be of animal origin, such as, for example, collagen, elastin, fish flesh protein, fish gelatin, keratin or casein, of plant origin, like cereal, flower or fruit proteins such as for example the proteins derived from soya bean, sunflower, oats, wheat, maize, barley, potato, lupin, field bean, sweet almond, kiwi, mango or apple; they may also be proteins obtained from Chorella (unicellular algae), pink algae, yeast or silkweed. This hydrolysis is carried out, for example, by heating, to temperatures of between 60 and 130° C., a protein placed in an acidic or alkaline medium. This hydrolysis may also be carried out enzymatically with a protease, optionally combined with a post-alkaline or acid hydrolysis. When m is greater than 1, R₂ represents one and the same chain or else several chains characterizing different amino acids, depending on the protein hydrolysed and the degree of hydrolysis. The aminograms of a few proteins of plant origin are presented in the following table:

TABLE A
Origin of the protein
(proportions of amino acids expressed
in % by weight)

| | Oats | Soya bean | Wheat | Sunflower | Lupin | Potato | Field bean | Maize |
|---|---|---|---|---|---|---|---|---|
| Glycine | 6.9 | 4.2 | 3.2 | 6.2 | 0.9 | 4.8 | 4.0 | 2.4 |
| Alanine | 5.9 | 4.2 | 2.6 | 4.8 | 2.4 | 5.0 | 4.0 | 7.95 |
| Serine | 5.6 | 5.1 | 1.7 | 5.1 | 6.1 | 5.8 | 4.9 | 5.1 |
| Aspartic acid | 16.2 | 11.7 | 3.4 | 10.6 | 15.8 | 12.5 | 10.5 | 10.6 |
| Glutamic acid | 28.3 | 19.1 | 37.9 | 23.6 | 8.0 | 11.5 | 16.8 | 23.6 |
| Valine | 2.9 | 5.0 | 4.2 | 4.8 | 7.9 | 7.1 | 4.5 | 4.8 |
| Threonine | 3.1 | 3.9 | 2.7 | 4.4 | 8.1 | 6.1 | 3.6 | 4.4 |
| Arginine | 6.6 | 7.8 | 3.7 | 8.4 | 16.1 | 5.0 | 9.21 | 8.4 |
| Lysine | 3.6 | 6.2 | 1.9 | 3.2 | 7.1 | 7.8 | 6.5 | 6.2 |
| Proline | 4.7 | 5.4 | 11.7 | 3.0 | — | 5.1 | 4.4 | 3.0 |
| Leucine | 6.4 | 8.1 | 7.1 | 6.4 | 7.45 | 10.4 | 7.4 | 8.1 |
| Phenylalanine | 1.4 | 5.0 | 5.4 | 4.3 | 8.6 | 6.4 | 4.4 | 4.3 |
| Isoleucine | 2.2 | 4.8 | 3.7 | 4.1 | 8.7 | 6.1 | 3.9 | 4.1 |
| Histidine | 1.7 | 2.6 | 2.4 | 2.0 | — | 2.2 | 2.6 | 2.0 |
| Tyrosine | 1.5 | 3.5 | 3.1 | 2.7 | — | 5.7 | 3.6 | 2.7 |
| Methionine | 1.2 | 1.2 | 1.6 | 1.8 | 0.6 | 2.4 | 0.8 | 1.8 |
| Cysteine/ | 1.9 | 1.5 | 1.9 | 1.9 | — | 1.6 | 1.7 | 1.9 |
| cystine | | | | | | | | |
| Tryptophan | — | 1.0 | 1.0 | 1.3 | 1.2 | 1.4 | 1.2 | 1.3 |
| Ornithine | | | | | 0.4 | — | — | — |

The acylation reaction is known to persons skilled in the art. It is described, for example, in international application published under the number WO 98/09611. It is carried out either on an amino acid or on a mixture of amino acids. The acylating agent generally consists of an activated derivative of a carboxylic acid of formula R₁C(═O)—OH, such as a symmetric anhydride of this acid or an acid halide such as acid chloride or acid bromide. It may also consist of a mixture of activated derivatives of carboxylic acids derived from natural oils or fats of animal or plant origin such as copra, palm kernel, palm, soya bean, rapeseed or maize oils, or beef tallow, spermaceti oil or herring oil.

The subject of the invention is most particularly a composition as defined above, for which the compound of formula (I) is an N-lauroylamino acid or a mixture of N-cocoylamino acids. As example of such mixtures, there is PROTEOL™ SAV 50S or PROTEOL™) OAT marketed by the company SEPPIC.

The compounds of formula (IIa), corresponding to formula (II) as defined above, for which Z does not represent a hydrogen atom, are prepared by reacting alkoxylated and phosphated polysiloxanes, such as those whose preparation is described in American patents published under the numbers U.S. Pat. No. 5,070,171, U.S. Pat. No. 5,091,493, U.S. Pat. No. 5,093,452, U.S. Pat. No. 5,100,956, U.S. Pat. No. 5,149,765 or U.S. Pat. No. 5,243,028, with panthenol. One of these compounds is commercially available under the name PECOSIL™ SPP 50, called according to the INCI name: potassium dimethicone copolyol panthenyl phosphate.

According to a fifth particular aspect, the subject of the invention is a composition as defined above, in which the compound of formula (II) is panthenol. According to this variant, the composition may also comprise one or more surfactants with phosphate groups of the family of alkoxylated and phosphated polysiloxanes, such as those described in American patents published under the numbers U.S. Pat. No. 5,070,171, U.S. Pat. No. 5,091,493, U.S. Pat. No. 5,093,452, U.S. Pat. No. 5,100,596, U.S. Pat. No. 5,149,765 or U.S. Pat. No. 5,243,028 and more particularly one of the dimethicone copolyol phosphate salts marketed under the names PECOSIL™ PS-100, PECOSIL™ PS-200 or PECOSIL™ WDS-100.

According to a sixth particular aspect, the subject of the invention is a composition as defined above, in which the compound of formula (II) is potassium dimethicone copolyol panthenyl phosphate or PECOSIL™ SPP-50. According to this variant, the composition may also comprise one or more surfactants containing phosphate groups of the family of the alkoxylated and phosphated polysiloxanes, such as those described in American patents published under the numbers U.S. Pat. No. 5,070,171, U.S. Pat. No. 5,091,493, U.S. Pat. No. 5,093,452, U.S. Pat. No. 5,100,956, U.S. Pat. No. 5,149,765 or U.S. Pat. No. 5,243,028 and more particularly one of the dimethicone copolyol phosphate salts marketed under the names PECOSIL™ PS-100, PECOSIL™ PS-200 or PECOSIL™ WDS-100.

The composition which is the subject of the present invention is prepared by methods known to persons skilled in the art. In addition to the active ingredients, the composition according to the invention comprises inorganic or organic vehicles commonly used in the manufacture of compositions intended to be formulated as preparations for cosmetic and/or pharmaceutical use. There may be mentioned, for example, water or water-alcohol mixtures, such as aqueous solutions of ethanol, propanol or isopropanol. There may also be mentioned polyols such as propylene glycol, dipropylene glycol, butylene glycol, hexylene glycol, glycerin or 1,2-octanediol.

According to a preferred aspect of the present invention, the composition as described above comprises from 15% to 60%, more particularly from 20% to 40% by weight of at least one compound of formula (I) and from 10% to 40% by weight and preferably from 15% to 30% by weight of at least one compound of formula (II).

The subject of the invention is also the use of the composition as defined above for preparing cosmetic, dermocosmetic, dermopharmaceutical or pharmaceutical compositions. The cosmetic, dermocosmetic, dermopharmaceutical or pharmaceutical compositions thus prepared generally contain from 0.1% to 10% by weight and more particularly from 1% to 3% by weight of the composition as defined above. The cosmetic, dermocosmetic, dermopharmaceutical or pharmaceutical compositions possess in particular protective, nourishing, anti-stress, coating and/or restructuring action. They are more particularly intended for the treatment of the scalp, hair or hair folicles.

The subject of the invention is also the use of the composition as defined above for the preparation of compositions intended to be deposited, absorbed or impregnated onto, or by woven or nonwoven supports such as for example an item of clothing or underwear so that the latter offers a sensation of wellbeing to the person wearing it.

By virtue of its soothing properties, the composition according to the invention may be used in any products containing components which are irritant to a greater or lesser degree, so as to enhance their tolerance as, for example, in antidandruff products. The composition according to the invention may also be used in synergy with other products normally used for the preparation of topical products. This includes in particular soothing products such as alpha-bisabolol, liquorice derivatives such as glycyrrhetinic acid or its derivatives or allantoin, hyperoxygenated oils such as epaline, essential waxes, oils, products based on oligosaccharides, products based on peptides, extracts of plants such as for example extracts of cinnamon, of water lily flower, of Aloe vera or Centella asiatica, extracts of algae, mineral salts (or products based on minerals), products known for their anti-free-radical property such as polyphenols, glutathione, vitamins such as vitamin E or vitamin C, enzymes such as superoxide dismutase or glutathione peroxidase, products known for their anti-inflammatory properties, vitamins in general (or products based on vitamins), enzymes in general (or products based on enzymes), products having activity towards neuromediators.

Depending on the use envisaged, the composition as described above is used at different concentrations and in a formulation appropriate for this use. Such cosmetic, dermocosmetic, dermopharmaceutical or pharmaceutical compositions are normally provided in the form of aqueous solutions, dilute alcoholic solutions, oils or single or multiple emulsions, such as water-in-oil (W/O), oil-in-water (O/W) or water-in-oil-in-water (W/O/W) emulsions in which the oils are of a plant, mineral or synthetic nature such as for example silicone oils. As cosmetic formulation, there may be mentioned oils such as for example hair oil or creams, gels, milks, lotions, shower gels, gel creams, soaps, liquid soaps, syndets or shampoos.

Such formulations are known to persons skilled in the art; their preparations are described, for example, in patent applications published under the. numbers WO 92/06778, WO 93/28204, WO 95/13863, WO 95/35089 or WO 96/22109.

The subject of the invention is therefore also a cosmetic formulation which can be obtained by diluting from 1/10 to 1/20,000preferably from 1/10 to 1/100, the composition as described above, in one or more cosmetically acceptable excipients, and in particular a cosmetic formulation in the form of an oil-in-water emulsion having the appearance of a milk having a viscosity of less than 1 Pa.s comprising, as emulsifier, a self-emulsifying composition based on fatty alcohols.

As preferred emulsifying compositions, there may mentioned MONTANOV™ 68, MONTANOV™ 14, MONTANOV™ 82 and MONTANOV™ 202 or MONTANOV™ WO18, which are marketed by the company SEPPIC.

Depending on the character which it is possible to give the cosmetic formulation, it is possible, where appropriate, to add a reverse latex such as SEPIGEL™ 305, SEPIGEL™ 501, SIMULGEL™ 600 or SIMULGEL™ EG. The term dilution used in the preceding text covers, in its broadest meaning, all the steps which make it possible to pass from the composition as defined above to the cosmetic formulation intended to be marketed. In another preferred embodiment of the present invention, the cosmetic formulation is a soothing oil, cream or milk for treating the scalp. In another preferred embodiment of the present invention, the cosmetic formulation is a foam formula or a shampoo.

The following examples illustrate the invention without, however, limiting it.

### EXAMPLE 1

Preparation of a Composition According to the Invention and Demonstration of Its Properties

A) Preparation

A composition (A) according to the invention is prepared by mixing, with stirring, 75 grams of PROTEOL™ SAV 50, which is a mixture at about between 30% and 40% by weight of active substance, of N-cocoylamino acids with 25 grams of PECOSIL™ SPP 50, consisting at 100% of potassium dimethicone polyol panthenyl phosphate.

B) Demonstration of the Human Leukocyte Elastase Inhibiting Properties of the Composition According to the Invention

a) Principle of the Test

Human leukocyte elastase (HLE) is involved in a large number of inflammatory pathological conditions. This enzyme is in particular capable of degrading many macromolecules such as fibrous elastin, some types of collagen, proteoglycans and glycoproteins. For this reason, HLE constitutes one of the links in the chain of reactions accompanying the inflammatory phenomenon. The blocking of this enzyme by an “anti-elastase” effect therefore makes it possible to prevent the degradation of the abovementioned molecules and therefore to inhibit the inflammatory process. The “anti-elastase” properties of a given product can be demonstrated by a test in vitro using a substance which is degraded by HLE while becoming coloured, in which the variations in colour are determined by spectrophotometry. The substance used in the present test is N-methoxysuccinyl-alanine-proline-valine-para-nitroanilide, a normally colourless substance which releases, upon hydrolysis by HLE, para-nitroanilide, whose kinetics of appearance is monitored by spectrophotometry at 410 nm. The reaction is carried out in a spectrophotometer thermostated at 25° C., equipped with a sample changer. All the kinetics are performed at least three times, the mean and the standard deviation then being calculated for the three values obtained. The presence of a molecule with “anti-elastase” activity results in limiting of the appearance of the coloured product and this effect is calculated with respect to a standard curve obtained in the absence of the said molecule. There is thus a correlation between the percentage inhibition of the appearance of the coloured product by the test compound and the percentage inhibition of HLE. The percentage inhibition thus calculated is also representative of the soothing activity of the test compound.

b) Trial

The following aqueous solutions are prepared:

Solution 1: aqueous solution containing 2.5% active substance, PECOSIL™ SPP 50:

Solution 2: aqueous solution containing 0.015% active substance, PROTEOL™ SAV SOS;

Solution 3: aqueous solution containing 2.5% active substance, PECOSIL™ SPP 50 and containing 0.015% active substance, PROTEOL™ SAV 50S.

The percentage inhibition of the appearance of para-nitroanilide is measured for each of these three solutions using a spectrophotometer at 410 nm according to the protocol described in the preceding paragraph.

The results are presented in the following table:

| | % inhibition |
|---|---|
| Solution 1 | 30% |
| Solution 2 | 36% |
| Solution 3 | 89% |

These results show that at concentrations for which PROTEOL™ SAV 50S (solution 2) and PECOSIL™ SPP 50 (solution 1) have a limited activity (36% and 30%), the combination of the two products unexpectedly exhibits a higher activity (89% inhibition).

C) Demonstration of the Peroxide Formation Inhibiting Properties of the Composition According to the Invention

a) Principle of the Study “Ex Vivo”

The protective effect of composition (A) is evaluated by determining the amount of peroxides present at the surface of locks of hair subjected to UVA-type ultraviolet radiation at a power of 25 Joule/cm², the composition (A) having been applied before irradiation or after irradiation. The lipid peroxides are assayed by analysing the fluorescence induced by the oxidation of the dichlorofluorescein which they cause. The amount of peroxides present at the surface of the hair is expressed in FU units per mg of hair.

b) Trial

This study is carried out with 40 locks of hair taken from healthy volunteers, previously made lipid-free in ethanol at 70%, and then rinsed. They are distributed into four batches of 10 locks called batches A, B, C and D. The batches of locks are subjected to the following treatments:

The locks of batch A are soaked in water for 10 minutes and then dried in open air for 30 minutes.

The locks of batch B are soaked in water for 10 minutes, dried in open air for 30 minutes and then placed in a VILBER LOURMAT™ irradiation device and subjected to UVA radiation of 25 Joule/cm².

The locks of batch C are soaked in an aqueous solution containing 1% by weight of composition (A) for 10 minutes, dried in open air for 30 minutes and then placed in a VILBER LOURMAT™ irradiation device and subjected to UVA radiation of 25 Joule/cm².

The locks of batch D are soaked in water for 10 minutes, dried in open air for 30 minutes, placed in a VILBER LOURMAT™ irradiation device and subjected to UVA radiation of 25 Joule/cm² and then they are soaked in an aqueous solution containing 1% by weight of composition (A) for 10 minutes and dried in the open air for 30 minutes.

The peroxides are assayed 24 hours after the irradiation. All the locks are rinsed and then weighed and brought into contact with dichlorofluorescein. The fluorescence is measured with Fluoroskan™. It is then weighted relative to the mass of hair.

The results are presented in the following table:

| | Batch A | Batch B | Batch C | Batch D |
|---|---|---|---|---|
| Amount of lipid | 354 | 672 | 535 | 447 |
| peroxides (in FU/mg | | | | |
| of hair) | | | | |
| Relative amount | 52.7 | 100 | 79.6 | 66.5 |
| (base 100 = batch B) | | | | |

These results demonstrate the protective and restorative actions of composition (A) in relation to the action of the UVA-type ultraviolet rays.

D) Demonstration of the Anti-Free-Radical Properties of the Composition According to the Invention

a) Principle of the Study

The determination of the anti-free-radical effect is based on the capacity which the molecule to be studied has to inhibit or reduce the rate of reduction of cytochrome C, when it is added to the reaction medium. The superoxide anion is formed by the action of xanthine oxidase on xanthine. It induces, in the absence of a molecule capable of capturing it, the reduction of cytochrome C. The appearance of reduced cytochrome C is monitored in a spectrophotometer at 550 nm, in the presence (Trial) and in the absence (Control) of anti-free-radical molecules.

b) Trial

The study consists in comparing the anti-free radical activity of composition (A) with that of vitamin C (ascorbic acid) and that of panthenol or D(+)-2,4-dihydroxy-N-(3-hydroxypropyl)-3,3-dimethylbutanamide, which is a compound commonly used for protecting the hair.

The reaction is carried out in a spectrophotometer thermostated at 25° C. and provided with a sample changer. All the kinetics are determined at least three times; the mean and the standard deviation are calculated for the three values obtained. A percentage inhibition of the rate of appearance of the coloured product (corresponding to the quantity of free superoxide anion) is therefore calculated for each active agent tested. The calculation is performed relative to the rate of appearance of the coloured product in the control (without active agent). The percentage inhibition of the appearance of the coloured product by the active agent therefore corresponds to the percentage inhibition of the superoxide anion. The results are presented in the following table:

| | | % anti-free |
|---|---|---|
| | | radical |
| Products | Concentrations tested | inhibition |
| Vitamin C | 0.46% (as active | 94% |
| | substance) | |
| Panthenol | 0.46% (as active | 0% |
| | substance) | |
| Composition (A) | 0.46% (as active | 94% |
| | substance) | |

These results demonstrate an anti-free radical activity of composition (A) of the same order as that of vitamin C, unlike that which is nonexistent for panthenol.

E) Demonstration of the Cell Division Stimulating Effect of the Composition According to the Invention

a) Principle of the Study “In Vitro”

The effect of the active agents on cell division is measured by a fluorimetric assay of the content of DNA in normal human keratinocytes subjected to a high thermal stress (20 minutes at 50° C.). The quantity of DNA present in the cells determines their capacity to divide. The cells are used at 60% confluence. They are incubated for 24 hours in the presence of the active agents. Cell division is determined by assaying the quantity of DNA present per well.

b) Trial

Four samples A, B, C and D, of a culture normal human keratinocytes, are subjected to the following respective treatments:

Sample A: cells not treated and left at room temperature.

Sample B: cells not treated and heated at 50° C. for 20 minutes.

Sample C: cells left incubated for 24 hours with composition (A) (concentration: 4.75×10⁻⁶% by weight of active substance) and heated at 50° C. for 20 minutes.

Sample D: cells left incubated for 24 hours with panthenol (concentration: 12.5×10⁻⁶% by weight of active substance) and heated at 50° C. for 20 minutes.

The DNA in each of the samples is then assayed.

The results, expressed in μg/well, are presented in the following table:

| | Sample A | Sample B | Sample C | Sample D |
|---|---|---|---|---|
| Cell division | 4.5 | 3.7 | 5.7 | 4.3 |
| (in μg/well) | | | | |
| Relative level | 116.2 | 100 | 154.1 | 116.2 |
| (base 100: | | | | |
| batch B) | | | | |

These results demonstrate the capacity of composition (A) to stimulate cell division so as to surpass the harmful effects of heat, whereas panthenol only compensates for these effects.

F) Demonstration of the Hair Root Protein Metabolism Protecting Effect of the Composition According to the Invention

a) Principle of the Study “In Vitro”

The effect of the protection of the metabolism of proteins is determined by colorimetric assay of the protein content of the cells (expressed in μg/ml) after incubating for 24 hours in the presence of the active agents. The cells are used at 60% confluence.

b) Trial

Five samples E, F, G. H and I, of a culture normal human keratinocytes, are left incubated for 24 hours in the presence or otherwise of one of the following solutions 1, 2 or 3:

Solution 1: aqueous solution containing 1.25×10⁻⁵% as active substance, of PECOSIL™ SPP 50;

Solution 2: aqueous solution containing 1.125×10⁻⁵% as active substance, of PROTEOL™ SAV 50S;

Solution 3: aqueous solution containing 1.25×10⁻⁵% as active substance, of PECOSIL™ SPP 50 and containing 1.125×10⁻⁵% as active substance, of PROTEOL™ SAV 50S.

They are then subjected or otherwise to a temperature of 50° C. for 20 minutes:

Sample A: cells not treated and left at room temperature.

Sample F: cells not treated and heated to 50° C.

Sample G: cells treated with solution 1 and heated to 50° C.

Sample H: cells treated with solution 2 and heated to 50° C.

Sample I: cells treated with solution 3 and heated to 50° C.

The DNA in each of the samples is finally assayed.

The results, expressed in μg/ml, are presented in the following table:

| Sample | E | F | G | H | I |
|---|---|---|---|---|---|
| Level of cellular | 7.7 | 5.6 | 5.7 | 8.2 | 10.5 |
| proteins in μg/ml | | | | | |
| Relative level | 138 | 100 | 102 | 146 | 188 |
| (base 100: sample F | | | | | |

These results demonstrate the capacity of composition (A) to stimulate protein metabolism in the keratinocytes in order to counteract the negative effects of heat, whereas the compounds of formula (II) alone are inactive and the compounds of formula (I) alone are moderately active.

G) Demonstration of the Hair Protein Degradation Inhibiting Effect of the Composition According to the Invention

a) Principle of the Study “Ex Vivo”

The protective effect and the preventive action of composition (A) are evaluated by measuring the intrinsic fluorescence of the tryptophan of the keratin of locks stressed either by ultraviolet radiation or by heat. Protein degradation is characterized by a reduction in the intrinsic natural fluorescence of the tryptophan.

b) Trial

This study is carried out with 40 locks of hair taken from healthy volunteers, previously made fat-free in ethanol at 70%, and then rinsed. They are divided into five batches of 10 locks, called batches J, K, L, M and N, which are treated as follows:

The locks of batch J are soaked in water for 10 minutes, dried in open air for 30 minutes (untreated batch does not suffer any stress).

The locks of batch K are soaked in water for 10 minutes, dried in open air for 30 minutes and then placed in a VILBER LOURMAT™ irradiation device and subjected to UVA radiation of 25 Joule/cm².

The locks of batch L are soaked in water for 10 minutes, dried in open air for 30 minutes and they are then subjected to an air stream at 90° C. for 1 hour and they are then left at room temperature for 15 minutes.

The locks of batch M are soaked in an aqueous solution containing 1% by weight of composition (A) for 10 minutes, dried in open air for 30 minutes and then placed in a VILBER LOURMAT™ irradiation device and subjected to UVA radiation of 25 Joule/cm².

The locks of batch N are soaked in an aqueous solution containing 1% by weight of composition (A) for 10 minutes, dried in open air for 30 minutes and they are then subjected to an air stream at 90° C. for 1 hour and they are then left at room temperature for 15 minutes.

The fluorescence intensity is measured by means of a CD60 DESAGA™ spectrophotrometer. The fluorescence is acquired on a fixed and determined hair surface (350 mm²) and in the longitudinal direction (equivalent to the direction of the root towards the tip). The fluorescence intensity value is determined in arbitrary units expressed relative to the mass of hair.

The results, expressed as degradation of intrinsic fluorescence of the tryptophan per gram of hair, relative to the untreated and unstressed (Δ=0) batch J, are presented in the following table:

| | Batch J | Batch K | Batch L | Batch M | Batch N |
|---|---|---|---|---|---|
| Δ (in AU/g) | 0 | −193 | −163 | −73 | −107 |
| Relative level of | — | 100 | — | 38 | — |
| degradation (base | | | | | |
| 100: batch K) | | | | | |
| Relative level of | — | — | 100 | — | 66 |
| degradation (base | | | | | |
| 100: batch L) | | | | | |

These results demonstrate the capacity of composition (A) to slow down the degradation of the keratin in hair subjected to a thermal stress or a photochemical stress.

H) Demonstration of the Inhibitory Effect of the Composition According to the Invention on the Formation of Flakes at the Surface of the Hair

a) Principle of the Study “Ex Vivo”

The capacity of an aqueous solution containing 3% by weight of composition (A), called solution 4, to prevent the formation of flakes on the surface of the hair was tested on damaged hair subjected to thermal stress.

This property is demonstrated by observing the surface of treated and untreated hair by scanning electron microscopy.

b) Trial

Hair is taken from healthy volunteers. 5 hair strands from each volunteer are soaked for 10 minutes either in solution 4 or in water (placebo). All the hair strands are dried in open air and they are then subjected to an air stream at 90° C. for 1 hour and then left at room temperature for 15 minutes and observed by scanning electron microscopy and by taking photographs.

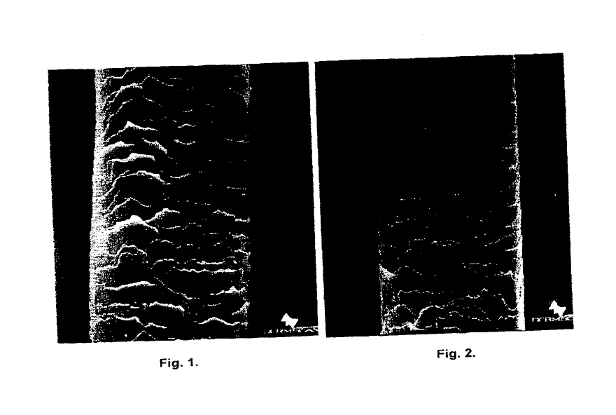

FIGS. 1 and 2 demonstrate the protective effect of the composition of the invention in relation to the formation of flakes at the surface of the hair.

The following examples illustrate the use of composition (A) for preparing cosmetic formulations.

### EXAMPLE 2

Thermoactive Hair Lotion

| Formula | |
|---|---|
| Butylene glycol: | 3.0% |
| SIMULSOL ™1293: | 3.0% |
| Composition (A): | 1.0% |
| Lactic acid: | QS pH = 6 |
| SEPICIDE ™ HB: | 0.2% |
| SEPICIDE ™ Cl: | 0.3% |
| Perfume: | 0.3% |
| Water: | QS 100% |

### EXAMPLE 3

Protective and Relaxing Shampoo

| Formula | |
|---|---|
| Amonyl ™ 675 SB: | 5.0% |
| Sodium lauryl ether sulphate at 28%: | 35.0% |
| Composition (A): | 1.0% |
| Capigel ™ 98: | 3.0% |
| SEPICIDE ™ HB: | 0.5% |
| SEPICIDE ™ Cl: | 0.3% |
| Sodium hydroxide: | QS pH = 7.2 |
| Perfume: | 0.3% |
| Colorant (FDC blue 1/yellow 5) | QS |
| Water: | QS 100% |

Characteristics

The shampoo obtained has a green clear appearance. Its pH is approximately equal to 7.2 and its viscosity is equal to 1000 cps (BROOKFIELD™ LVT: M4 V6)

### EXAMPLE 4

“Leave-On” Protector; Anti-Stress Care for Hair

| Formula | |
|---|---|
| KETROL ™ T: | 0.5% |
| Composition (A) | 3.0% |
| Butylene glycol: | 5.0% |
| DC 1501: | 5.0% |
| SIMULGEL ™ EG: | 4.0% |
| SEPICIDE ™ HB: | 0.5% |
| SEPICIDE ™ Cl: | 0.3% |
| Perfume: | 0.3% |
| Water: | QS 100% |

Characteristics

The care product obtained is in the form of an opaque gel. Its pH is approximately 6.5 and its viscosity is equal to 40,000 cps (BROOKFIELD™ LVT: M4 V6).

### EXAMPLE 5

Restructuring “Rinse Off” Cream Mask for Stressed and Brittle Hair

| Formula | |
|---|---|
| KETROL ™ T: | 0.5% |
| Composition (A): | 3.0% |
| Butylene glycol: | 3.0% |
| SIMULGEL ™ EG: | 1.0% |
| MONTANOV ™ 82: | 3.0% |
| Jojoba oil: | 1.0% |
| LANOL ™ LP: | 6.0% |
| AMONYL ™ DM: | 1.0% |
| LANOL ™ 99: | 5.0% |
| SIMULGEL ™ EG: | 4.0% |
| SEPICIDE ™ HB: | 0.3% |
| SEPICIDE ™ Cl: | 0.2% |
| Perfume: | 0.2% |
| Water: | QS 100% |

Characteristics

The mask obtained is in the form of a cream. Its pH is approximately equal to 6.2 and its viscosity is equal to 40,000 cps (BROOKFIELD™ LVT: M4 V6).

The definitions of the commercial products used in the examples are the following:

SIMULSOL™ 1293 is hydrogenated and ethoxylated castor oil, with an ethoxylation value equal to 40, marketed by the company SEPPIC.

SEPICIDE™ HB is a preserving mixture comprising phenoxyethanol, methylparaben, ethylparaben, propylparaben and butylparaben, marketed by the company SEPPIC.

SEPICIDE™0 Cl is imidazolidinylurea, marketed by the company SEPPIC.

CAPIGEL™ 98 is a liquid thickener based on acrylate copolymer marketed by the company SEPPIC.

AMONYL™ 675SB is a sulphobetaine marketed by the company SEPPIC.

SIMULGEL™ EG is a reverse latex of copolymer (INCI name: sodium acrylate/sodium acryloyldimethyltaurate copolymer and isohexadecane and polysorbate 80) marketed by the company SEPPIC.

KETROL™ T is xanthan gum marketed by the company KELCO.

LANOL™ 99 is isononyl isononanoate marketed by the company SEPPIC.

DC1501 is a mixture of cyclopentasiloxane and dimethiconol marketed by the company DOW CHEMICAL.

MONTANOV™ 82 is an emulsifying agent based on cetearyl alcohol and cocoylglucoside.

What is claimed:

1. A method of protecting human hair against damage, comprising applying to human hair in need of the same a composition comprising as active ingredients an N-lauroylamino acid or a mixture of N-cocoylamino acids, and potassium dimethicone copolyol panthenyl phosphate.

2. A method as claimed in claim 1, wherein said composition comprises also at least one vehicle selected from the group consisting of water, aqueous solutions of ethanol, propanol or isopropanol, propylene glycol, dipropylene glycol, butylene glycol, hexylene glycol, glycerin and 1,2-octanediol.

3. A method as claimed in claim 1, wherein in said composition, said N-lauroylamino acid or a mixture of N-cocoylamino acids is present in an amount of 15% to 60% by weight and said potassium dimethicone copolyol panthenyl phosphate is present in an amount from 10% to 40% by weight.

4. A method as claimed in claim 1, wherein, in said composition, said N-lauroylamino acid or a mixture of N-cocoylamino acids is present in an amount of 20% to 40% by weight and said potassium dimethicone copolyol panthenyl phosphate is present in an amount from 15% to 30% by weight.

5. A method as claimed in claim 1, wherein said composition is present in an amount between 0.1% and 10% by weight in admixture with a cosmetically-acceptable excipient.

6. A method as claimed in claim 5, wherein said composition is present in an amount of 1% to 3% by weight.
